(19) United States
(12) Patent Application Publication
(10) Pub. No.: US 2025/0205125 A1
(43) Pub. Date: Jun. 26, 2025

(54) Skin Protection Compositions

(71) Applicant: Jennifer Leebow, Miami beach, FL (US)

(72) Inventor: Jennifer Leebow, Miami beach, FL (US)

(21) Appl. No.: 19/002,119

(22) Filed: Dec. 26, 2024

Related U.S. Application Data
(60) Provisional application No. 63/614,711, filed on Dec. 26, 2023.

(51) Int. Cl. A61K 8/06; A61Q 17/04

(52) U.S. Cl. CPC A61K 8/06; A61Q 17/04; A61K 2800/5922; A61K 2800/92

(57) ABSTRACT

Skin protection formulations having unexpectedly synergistic effects are disclosed. Such formulations include a nutritional supplement, a cream, and serum formulation. The selective use of these elements in a predetermined schedule allow the body of the user to have increased photoprotective properties. The nutritional supplement and the serum can be used without the cream, although use of the cream is beneficial to further enhance skin protection against the harmful effects of UV radiation, HEV light, and environmental stressors like air, soil, and water pollution.

### CROSS-REFERENCE TO RELATED APPLICATIONS

This application claims the benefit of the filing date of U.S. Provisional Patent Application No. 63/614,711 filed Dec. 26, 2023, the disclosure of which is hereby incorporated herein by reference.

### FIELD OF THE TECHNOLOGY

The embodiments relate generally to skin care, and more specifically, to oral and topical compositions for protecting human skin.

### BACKGROUND

Ultraviolet (UV) radiation, environmental toxins, high-energy visible light (HEV), and additional environmental stressors such as air pollution, water pollution, and soil pollution collectively pose a multifaceted threat to human skin, giving rise to a dual classification of skin damage; acute and chronic.

Acute effects encompass a range of conditions, including necrosis, erythema, and inflammation, triggered not only by high-energy UV photons but also by environmental factors like air pollutants (e.g., nitrogen dioxide), water pollutants, and soil contaminants. These environmental factors can initiate acute skin damage by directly impacting the skin's protective barrier.

Chronic consequences extend beyond UV radiation and encompass the persistent assault from environmental stressors. Chronic skin damage includes not only photoaging, characterized by the emergence of wrinkles and alterations in skin pigmentation but also additional damage inflicted by prolonged exposure to air pollution, water pollutants, soil contaminants, and HEV light. These factors collectively contribute to chronic skin damage, which elevates the risk of skin cancer.

The primary lines of defense against UV damage are melanin and trans-urocanic acid, which are distributed in the stratum corneum. UV rays that pass beyond these lines of defense can lead to oxidative damage. However, cells detect changes due to UV rays as early as possible and initiate cell signaling processes to prevent the occurrence of damage and repair the already occurred damage.

Overexposure to UV radiation, particularly the UVB component, causes erythema, edema, hyperplasia, hyperpigmentation, photoaging, immunosuppression, and skin cancer. Additionally, overexposure of the skin to UV rays stimulates the production of reactive oxygen species (ROS); increases oxidative damage of biomolecules, such as lipids, nucleic acids, and proteins; and decreases endogenous antioxidants in the cutaneous tissues. Approximately 9-14% of solar UVB rays reach the dermis in the skin and can induce inflammatory responses, such as erythema and edema. Sunburn reactions are mediated by the tumor suppressor p53. The p53 arrests cell cycle, allowing cells to properly repair the damaged DNA or to remove the damaged cells, thereby reducing the risk of cancer development. Previous studies have demonstrated that exogenous antioxidants can prevent photocarcinogenesis.

Skin photoaging involves alterations in the extracellular matrix composition of the dermis. UV rays induce and activate matrix metalloproteinases (MMPs), a group of zinc endopeptidases that degrade extracellular matrix macromolecules, including type I collagen. MMPs secreted from both the dermal fibroblasts and epidermal keratinocytes participate in collagen metabolism in the skin. MMPs play key roles in connective tissue remodeling in UV-exposed skin and cause wrinkles and other phenotypes in photo-aged skin. Gene expression of MMPs, such as MMP-1, -2, -3, and -9, are upregulated in UV-exposed human dermal fibroblasts. The cell signaling pathways involve UV-induced activation of cytokine receptors and the subsequent activation of mitogen-activated protein kinases (MAPK), such as extracellular signal-regulated kinase, c-Jun-N-terminal kinase, and p38 kinase. The promoters of the MMP-1 and MMP-3 genes can be transactivated by activator protein-1 (AP-1) complexes. Although the initial events of this signal cascade are not fully understood, evidence suggests that UV-damaged DNA acts as a trigger that initiates this process.

Keratinocytes account for 95% of the mass of cells in the human epidermis and play an important role in maintaining skin homeostasis, through both their autocrine and paracrine effects. Under normal conditions, the constitutive production of cytokines and other soluble factors in human keratinocytes is low; however, various stimuli, such as UV rays and endotoxins, can trigger the expression of pro-inflammatory cytokines. Certain cytokines or cell components that are secreted from UVB-irradiated epidermal keratinocytes can regulate gene expression of dermal fibroblasts through paracrine effects. For example, interleukin (IL)-1B, tumor necrosis factor (TNF)-a, and stratifin are released from UV-exposed keratinocytes and stimulate MMP-1 expression in fibroblasts.

Apoptotic cell death is involved in skin photoaging, and typically involves changes in the expression of the pro-apoptotic and anti-apoptotic members of the Bcl-2 protein family, UV-induced apoptosis is mediated by caspases in keratinocytes. UV rays induce apoptosis of keratinocytes via intrinsic pathways, involving direct DNA damage; extrinsic pathways, involving activated cell membrane death receptors; and other ROS-mediated pathways. Apoptosis can be detected using various markers, including DNA laddering, changes in the expression of pro-apoptotic and anti-apoptotic members of the Bcl-2 protein family, and activation of caspases.

The distinction between acute and chronic skin damage hinges not only on the energy levels of incident UV radiation but also on the combined effects of various environmental factors. While high-energy UV photons cause acute damage, the cumulative biological effects of oxidative stress brought about by UV radiation, HEV light, and environmental stressors like air, soil, and water pollution are instrumental in causing chronic skin damage.

Oxidative stress, triggered by the combined influence of these factors, plays a central role in skin damage. It leads to the generation of reactive oxygen species (ROS) within the skin. These ROS can initiate a cascade of harmful biological reactions, including the deterioration of essential extracellular matrix components like collagen, cellular apoptosis, damage to plasma membranes, direct DNA damage, and an elevated risk of mutagenesis.

Traditional skin protection strategies, while effective to some extent, have seen limited innovation over the years. The last notable milestone in sunscreen innovation was the FDA's approval of new active ingredients in 1999. However, to comprehensively address the diverse causes of ROS and oxidative stress, a more multifaceted approach to skin protection is needed, taking into account not only UV radiation but also the broad spectrum of environmental factors and their cumulative effects on skin health.

Therefore, a need exists to overcome the problems with the prior art as discussed above, and particularly for a more effective and efficient way of providing protection for the skin.

### SUMMARY

The present disclosure provides for a method of providing photoprotection to a human user. In some embodiments, the method comprising the steps of first orally consuming, by the human user, one or more tablets configured to promote the human user's systemic protection against the effects of radiation in the ultraviolet, visible, and infrared spectra. Then, the method proceeds to the step of applying topically, by the human user, a serum configured to synergistically to promote the human user's systemic protection against the effects of radiation in the ultraviolet, visible, and infrared spectra in combination with the one or more tablets. These steps are repeated by the human user once per day for a predetermined amount of time.

Embodiments of this method exist where users also repeat the step of topically applying a cream configured to absorb an amount of radiation in the ultraviolet, visible, and infrared spectra. In some embodiments, the tablet includes up to 500 mg, up to 1000 mg, or up to 2,000 mg of methylsulfonylmethane, up to 150 mg, up to 300 mg, or up to 600 mg of bamboo extract, up to 150 mg, up to 300 mg, or up to 600 mg of fucoidan, up to 125 mg, up to 250 mg, or up to 500 mg of purslane extract, up to 125 mg, up to 250 mg or up to 500 mg of polypodium leucotomos, up to 60 mg, up to 125 mg, or up to 250 mg of L-glutathione, up to 60 mg, up to 125 mg, or up to 250 mg of rosemary powder, and up to 60 mg, up to 125 mg, or up to 250 mg of marigold extract. Embodiments exists where said tablet also includes 50 mg to 200 mg, 50 mg to 150 mg, or 50 mg to 100 mg of Beta Carotene 0.1% to 5%, 15 mg to 70 mg, or 15 mg to 60 mg, or 15 mg to 50 mg of niacinamide, 10 mg to 50 mg, 10 mg to 40 mg, or 10 mg to 30 mg of resveratrol 10% to 30% 10 mg to 50 mg, 10 mg to 40 mg, or 10 mg to 30 mg of red panax ginseng, 10 mg to 50 mg, 10 mg to 40 mg, or 10 mg to 30 mg of green tea 90% to 99% polyphenols, 10 mg to 40 mg, 10 mg to 30 mg, or 10 mg to 20 mg of selenium citrate 0.1%-5%, 5 mg to 30 mg, 10 mg to 20 mg, or 15 to 20 mg vitamin E, 5 mg to 30 mg, 10 mg to 20 mg, or 15 mg to 20 mg of pine mark extract, 5 mg to 30 mg, 10 mg to 20 mg, or 15 mg to 20 mg of mulberry fruit powder, 5 mg to 20 mg, 5 mg to 15 mg, or 5 mg to 10 mg of vitamin D3, 5 mg to 20 mg, 5 mg to 15 mg, or 5 mg to 10 mg of apigenin 90% to 99%, 1 mg to 10 mg, or 1 mg to 5 mg, or 2.5 mg to 5 mg of lycopene 1%-20%, 1 mg to 10 mg, or 1 mg to 5 mg, or 2.5 mg to 5 mg of quercetin, and 1 mg to 5 mg or 1 mg to 4 mg, 1 mg to 3 mg, or 1 mg to 2.5 mg of luteolin.

Embodiments of the serum exist where the serum includes glycerin, squalane, resveratrol, bakuchiol, silybum marianum fruit extract, niacinamide, mixed tocopherols, kojic acid, sodium ascorbyl phosphate, sodium hyaluronate, chondruis crispus extract, tumeric extract, licorice extract, green tea extract, brazilian bamboo extract, pine bark extract, red panax ginseng extract, marigold extract, beta carotene, calcium ketogluconate, tetrasodium glutamate diacetate, rosemary extract, caesalpinia spinosa gum, acacia senegal gum, xanthan gum, phenethylalcohol, and ethylhexyglycerin. Embodiments of the serum may also include water or purified water.

In some embodiments of the cream in accordance of the present disclosure, the cream includes zinc oxide up to 25% or up to 12%, C₁₂-C₁₅ alkyl benzoate, one or more jojoba esters, one or more triglycerides, polyglyceryl-4 diisostearate/polyhydroxystearate/sebacate, propanediol, niacinamide, methylpropanediol, coco-caprylate, polyglyceryl-2, Helianthus annuus extract R, rosemary extract, tocopherol, allantoin, Oryza sativa bran extract, caprylyl glycol, polyglyceryl-3, hydrogenated lecithin, and tridecyl salicylate. Embodiments of the cream can also include silica, nylon-12, bisabolol, tetrasodium glutamate diacetate, phenylpropanol, sodium chloride, and sodium hydroxide.

An embodiment of the nutritional supplement comprises the following ingredients, each present by weight percentage, including natural astaxanthin of 2% concentration (0.000%+20%), bamboo extract (0.0%+20%), glutathione (0.0%+20%), niacinamide (0.0%+20%), methylsulfonylmethane (0.0%+20%), rosemary powder (0.0%+20%), fucoidan powder (0.0%+20%), vitamin D3 (0.0%+20%), vitamin E (0.0%+20%), selenium citrate 0.5% (0.0%+20%), polygonum extract containing 20% resveratrol (0.0%+20%), pine bark extract (0.0%+20%), mulberry fruit powder (0.0%+20%), red panax ginseng (0.0%+20%), green tea containing 98% polyphenols (0.0%+20%, polypodium leucotomos (0.0%+20%), lycopene 10% (0.0%+20%), beta carotene 1% (0.0%+20%), apigenin 98% (0.0%+20%), quercetin (0.0%+20%), luteolin (0.0%+20%), purslane (0.0%+20%), and marigold extract 2:1 (0.0%+20%), all contributing to its skin protection properties.

Implementations may include one or a combination of any two or more of the aforementioned features or embodiments.

Other features, aspects, implementations, and advantages will become apparent from the descriptions and the claims.

### DETAILED DESCRIPTION

The various embodiments of the skin protection compositions can be broken up into three general categories: serums, oral nutritional supplements, and creams. These serums, supplements, and creams can be used individually but provide unexpectedly beneficial synergistic photoprotective effects when used in combination.

Reference will now be made in detail to each embodiment of the present disclosure. Such embodiments are provided by way of explanation of the present disclosure, and are not intended to be limited thereto in any manner whatsoever. In fact, those of ordinary skill in the art may appreciate upon reading the present specification that various modifications and variations can be made thereto.

While embodiments are described, modifications, adaptations, and other implementations are possible. For example, the methods described herein may be modified by substituting, reordering, or adding additional stages or components to the disclosed methods and devices.

The embodiments described herein enhance and supplement the body's inherent capability to combat the harmful effects of ultraviolet (UV) radiation, blue light exposure, and environmental factors. These embodiments operate in synergy with established physical skin protection methods. Specifically, they augment the body's basal antioxidant threshold, thereby enhancing its ability to respond to oxidative damage induced by UV radiation, HEV light, and environmental stressors like air, soil, and water pollution and heightened oxidative stress. The disclosed embodiments extend beyond conventional physical skin protection by introducing a proprietary blend comprising a combination of clinically validated ingredients at scientifically established dosages. This blend elevates the body's innate capacity to counteract free radical damage and oxidative stress resulting from exposure to UV radiation, HEV light, and environmental stressors like air, soil, and water pollution, consequently reducing the overall lifetime risk of adverse outcomes attributed to said harmful effects.

The disclosed embodiments encompass a proprietary formulation and a daily regimen aimed at augmenting the body's capacity to combat free radical damage and minimize oxidative stress within and on the skin. This regimen advocates oral supplementation in conjunction with the use of a topical serum, following a specific protocol.

A notable advantage of the disclosed embodiments lies in the utilization of a proprietary blend encompassing complex vitamins, minerals, and extracts in the oral nutritional supplement, which aligns with embodiments of the serum. This combination unexpectedly enhances the effectiveness of traditional topical SPF regimens, providing a comprehensive approach to skin protection.

The nutritional supplement may be provided in powder form to consumers. Said powder may be ingested by mixing with a liquid such as water or milk. The nutritional supplement may be provided in a solid capsule or tablet form, or may be in the form of a powder contained within a hollow capsule. The design of the capsule, including its composition, shell thickness, and any special coatings, can affect the release rate and absorption characteristics of the nutritional supplement. Some capsules are designed for immediate release, while others are formulated for sustained or delayed release to provide a longer-lasting effect.

In some embodiments, the cream includes zinc oxide. Embodiments exist where the zinc oxide is Kleair™ Zinc Oxide 12% or as high as 25%. The inclusion of zinc oxide within embodiments of the cream offers protections against a broad spectrum of ultraviolet radiation, including all UVA/UVB radiation. Additionally, the inclusion of zinc oxide helps the cream reduce exposure to pollution-induced free radicals to the user's skin.

Embodiments of the cream can also include C₁₂-C₁₅ alkyl benzoate, which is an ester of benzoic acid and C₁₂-C₁₅ alcohols, having a low molecular weight. When included in embodiments of the cream, any included sunscreen agents also present in the cream will have improved feel and softness to the user, and will be more easily dispersed across the user's skin. Embodiments exist where the cream include one or more ester blends made from the jojoba plant, also known as jojoba esters. These esters, when included in embodiments of said cream reduce the dryness of skin on which the serum is applied. This is due in part to the fact that these jojoba esters are similar to the oils naturally produced by human skin.

Embodiments of the cream can include one or more caprylic or capric triglycerides. These compounds are derived from coconut oil and glycerin and serve as an emollient when included in such embodiments. Moreover, the inclusion of these compounds also provide skin-replenishing properties to such embodiments of the cream. In some embodiments the cream may include an emulsifier such as polyglyceryl-4 diisostearate, polyhydroxystearate, or sebacate. In some embodiments, the cream may include propanediol, which may serve as a solvent, humectant, and emollient thereby enhancing the moisturizing properties of such creams.

In some embodiments of the cream, the cream includes an amount of niacinamide or other forms of vitamin B3. The inclusion of vitamin B3 allows said cream provides multiple benefits. First, such inclusion results in application of said cream to help build proteins in the skin and lock in moisture which prevent environmental damage to the user's skin. Additionally, such inclusion also provides for the cream to rebuild healthy skin cells in the user, while also shielding them from UV radiation. Moreover, the inclusion of vitamin B3 in the cream helps the skin upon which said cream is applied to develop ceramides which create a healthy lipid barrier, helping the skin retain moisture. The inclusion of niacinamide also helps the cream reduce inflammation in the skin from external factors such as UV radiation, environmental pollution, and toxins. These embodiments also help regulate the amount of oil the sebaceous glands produce, preventing an overproduction of sebum leading to acne and breakouts, also minimizing pore appearance. Additionally, these embodiments are effective in lightening hyperpigmentation by increasing collagen production in the user.

Embodiments of the cream also can include nylon-12 which serves as a texture enhancer and can serve as an absorbent. Silica may also be included to further enhance the texture. Embodiments of the cream may also include methylpropanediol as a solvent which also provides antimicrobial properties to the cream. In various embodiments, the cream also includes coco-caprylate which is an ester of C₈-C₁₀ fatty acids connected to C₁₂-C₁₈ fatty alcohols. The inclusion of these esters allows for the cream to absorb quickly while leaving a dry but silky finish on the user's skin.

Various embodiments of the disclosed cream can include rosemary (Rosmarinus Officinalis) extract. Rosmaric acid, which is included in this extract has an excellent bioavailability profile and has therapeutic effects on ultraviolet-induced skin aging. Embodiments of the cream exist where tocopherol, a form of vitamin E is included. Such inclusion allows the cream to better fight damage caused by free radicals and also to promote healing of the user's skin which, collectively, will diminish signs of aging. In some embodiments, allantoin is also included in the cream and serves as a skin conditioning agent. Rice (Oryza sativa) bran extract, can also be included in embodiment of the cream to allow the cream to boost the user's skin's natural regeneration process and protects from dehydration. Bisabolol, which is a scent ingredient naturally occurring in chamomile can also be included in the cream. Additionally, caprylyl glycol can be included in such creams to help condition the user's skin by providing antimicrobial activity.

In various embodiments, tetrasodium glutamate diacetate can be included in the cream and serves as a liquid chelating agent and a preservative. Embodiments of the cream can also include hydrogenated lecithin, which helps the cream have improved emollient properties by providing a high concentration of fatty acids. Tridecyl salicylate can be included to help soften the user's skin, and both sodium chloride and sodium hydroxide may also be included to help balance the overall pH of said cream.

Embodiments of the cream exist which include water, C₁₂-C₁₅ alkyl benzoate, zinc oxide, jojoba esters, caprylic/capric triglyceride, polyglyceryl-4 diisostearate/polyhydroxystearate/sebacate, propanediol, tridecyl salicylate, niacinamide, nylon-12, methylpropanediol, silica, coco-caprylate, polyglyceryl-2 dipolyhydroxystearate, sodium chloride, caprylyl glycol, bisabolol, allantoin, rice bran extract, lecithin, stearalkonium hectorite, polyglyceryl-3 diisostearate, polyglyceryl-3 polyricinoleate, phenylpropanol, sunflower extract, tocopherol, rosemary leaf extract, tetrasodium glutamate diacetate, and sodium hydroxide.

Embodiments of the cream also exist which include water, C₁₂-C₁₅ alkyl benzoate, zinc oxide, jojoba esters, caprylic/capric triglyceride, polyglyceryl-4 diisostearate/polyhydroxystearate/sebacate, propanediol, tridecyl salicylate, niacinamide, nylon-12, methylpropanediol, silica, coco-caprylate, polyglyceryl-2 dipolyhydroxystearate, sodium chloride, iron oxides, caprylyl glycol, bisabolol, allantoin, rice bran extract, lecithin, stearalkonium hectorite, polyglyceryl-3 diisostearate, polyglyceryl-3 polyricinoleate, phenylpropanol, sunflower extract, tocopherol, rosemary leaf extract, tetrasodium glutamate diacetate, and sodium hydroxide.

The present disclosure also contemplates multiple embodiments of a serum which can be topically applied to the user's skin. This serum, when combined with the cream and as described herein provides unexpectedly synergistic photoprotection to the user.

Embodiments of the serum can include glycerin which serves as a humectant, allowing the serum to pull water into the outer layer of a user's skin from the deeper layers as well as from the air. Embodiments of the serum also exist which include squalane, which provides for the serum to mimic the user's natural skin oils, making the serum serve as an effective emollient. Squalane also provide for the serum to have antioxidant and antitumor properties, thereby further protecting the user's skin from carcinogens. When coupled with the antioxidant effects of the cream, an unexpected synergistic effect is provided to the user. While squalane is produced naturally by human skin, this production is reduced with the age so its inclusion in embodiments of the serum is beneficial for users with reduced squalane production.

In embodiments of the serum, resveratrol is also included. Resveratrol can be obtained naturally from plants such as knotweed, and when included in the serum also allows for the serum to have improved antioxidant properties by further causing the serum to neutralize free radicals from the environment. Embodiments also exist where the serum includes bakuchiol, also known as Psoralea corylifolia. When included in embodiments of the serum, bakuchiol allows the serum to promote collagen production be stimulating collagen producing receptors in the skin. Additionally, in such embodiments, the serum will reduce the appearance of fine lines and wrinkles in the user, as well as restoring firmness, refine skin texture of the user, and will help even out the user's skin tone where the serum is applied. Moreover, these embodiments will also decrease inflammation in the user's skin caused by damage due to free radicals.

Embodiments of the serum exist where silybum marianum fruit extract is also included. In such embodiments, the serum will help increate the firmness and density of the user's skin while also providing further additional antioxidant properties. Additionally, these embodiments will help prevent premature aging of the user's skin by assisting with the prevention of fine lines, deep wrinkles, and the development of crow's feet in area's where the user's skin is thinning. Like some embodiments of the cream, embodiments of the serum exist which include niacinamide. When included in the serum, the inclusion of niacinamide will allow the serum to help build proteins in the user's skin and lock in moisture to prevent environmental damage. Additionally, these embodiments will also rebuild healthy skin cells in the user, while simultaneously shielding the user from UV radiation. Further, these embodiments will increase the photoprotective properties of a user's skin by promoting the development of ceramides which create a healthy lipid barrier, thereby aiding the user's skin's ability to retain moisture.

The inclusion of niacinamide in embodiments of the serum also imparts additional beneficial properties to the serum. Such inclusion helps reduce inflammation in the user's skin to due UV radiation, environmental pollution and toxins. When applied to the user's skin, these embodiments also help regulate the amount of oil produced in the sebaceous glands of the user and prevent any overproduction of sebum which can lead to acne and other breakouts while simultaneously minimizing the size of the user's pores. These embodiments also are effective in lightening any hyperpigmentation in the user's skin via the promotion of collagen production.

In embodiments of the serum, one or more mixed tocopherols are included to further improve the antioxidizing properties of the serum by stabilizing other elements which may be sensitive to oxidation. Kojic acid may also be included in embodiments of the serum, and in such embodiments the serum is able to even out the skin tone of a user by effectively targeting hyperpigmentation in the user's skin caused by UV damage. In such embodiments kojic acid also improves the antioxidizing properties of the serum, blocks tyrosinase thereby reducing melanin production in the skin, and further shields the user's skin against the damaging effects of UV radiation.

Embodiments of the serum exist which also include sodium ascorbyl phosphate further still enhancing the antioxidizing properties of the serum. Additionally, the inclusion of sodium ascorbyl phosphate also boosts the user's collagen production and evens the user's skin tone when the serum is applied, and also further improves the photoprotective properties of the serum while still being gentle on sensitive skin. Other forms of vitamin C can be included in the serum, but sodium ascorbyl phosphate is beneficial due to its shelf stability and is resistive to oxidation, resulting in a serum which provides a more consistent user experience. In some embodiments of the serum, sodium hyaluronate, a salt of hyaluronic acid is included to improve the serums resistance to oxidation. Sodium hyaluronate is beneficial due to its solubility in water, allowing it to penetrate deep within the user's skin, hydrating the lower layers of the user's skin. This deep moisturizing increases the firmness and density of the user's skin, while also minimizing fine lines and wrinkles present in the user's skin and serving as a humectant.

Various plant extracts are present in various embodiments of the serum. For example, chondruis crispus extract is also included in some embodiments of the serum. In such embodiments, the inclusion of this extract provides a source of antioxidants, natural polysaccharides, peptides, and amino acids, which collectively provide a wide range of benefits when applied to a user's skin such as providing protection against the visible effects of blue light exposure, damaging airborne pollutants, as well as other environmental stressors.

Turmeric extract, when applied topically as part of embodiments of the serum, can help improve the appearance of any skin tone irregularities present in the user's skin. Such irregularities include blotchiness, redness, and unevenness, dark spots, and other discolorations. As turmeric extract is rich in antioxidant compounds, including polyphenols, flavonoids, and curcuminoids, its inclusion in embodiments of the serum also provide enhanced antioxidizing properties to said serum. Other extracts can be included as part of embodiments of the serum such as licorice extract which allows the serum to brighten and even out the user's skin tone. Green tea extract may also be included in certain embodiments, and when included the serum neutralizes reactive oxygen species present in the environment while also upregulating antioxidant enzymes present in the user's skin.

Bamboo extract, such as Brazilian bamboo extract is beneficial to include in embodiments of the serum, as it provides a good source of flavonoids, and well as phenolic and polyphenol compounds which greatly improve the photoprotective and anti-inflammatory properties of the serum while promoting rejuvenation via cell growth. Specifically, the flavonoid quercetin increases the UV absorption profile of the serum while the polyphenols in the serum increase the UV absorption spectrum of the serum. Also, by decreasing oxidative stress and anti-inflammatory processes, all while stimulating DNA repair in the skin, bamboo extract provides additional benefits to the embodiments of the serum where it is present. These bamboo extracts, specifically extracts from the stem of bamboo, when included in the serum reduce oxidative stress by inhibiting mechanisms of reactive oxygen species produced by the Fenton reaction.

Additionally, embodiments of the serum which include these bamboo extracts will inhibit tyrosinase activity, thereby reducing melanin production. Moreover, such embodiments will reduce the expression levels and the mRNA transcription levels of TYR, TRP-1, and MITF. Such embodiments of the serum which include the bamboo extract will also further benefit the user's skin by interfering with the phosphorylation of protein kinase A, providing strong anti-melanogenic effects. These embodiments also provide a beneficial combinations of polyphenols, which further decrease oxidative stress and anti-inflammatory processes, and also stimulate DNA-repair and prevent skin damage. Moreover, the combination of polyphenols in these embodiments have absorb a broad spectrum of UV radiation, including UVA and UVB.

Embodiments of the serum can also include pine bark extract, which services as an antioxidant that stimulates collagen production while helping sooth any irritated skin of the user. The pine bark extract present in these embodiments binds with collagen and elastin, protecting skin from various harmful enzymes while also helping to rebuild elasticity. Additionally, these embodiments brighten the user's skin by preventing hyperpigmentation and also fight damaging effects of UV radiation not only by limiting exposure to UV radiation but also by repairing existing damage.

Red panax ginseng extract is also present in embodiments of the serum. As the red panax ginseng extract is higher in saponins than fresh and white ginseng, this extract is a more suitable ginseng extract than others for use in these embodiments. The antioxidant and anti-inflammatory properties imparted to the embodiments of the serum by this extract not only inhibit inflammation but also reduce oxidative damage to the user's skin cells. Embodiments of the serum exist which also include marigold (calendula) extract, which contains high polyphonic, flavonoid, and carotenoid contents, as well as lutein and zeaxanthin. The inclusion of these antioxidants further protect the user's skin from oxidative stress and promote cellular regeneration by scavenging ROS. These embodiments are effective against parakeratosis, and inhibit hyaluronidase, elastase, and MMP-1.

Moreover, the high content of terpenoids present in this extract also help boost collagen and elastin production, and also help defend the user's skin against the damage that free radicals from various environmental stressors.

When embodiments of the serum which contain beta carotene are applied topically, reduction of erythema induced by UV radiation is achieved. When combined with the cream, an unexpectedly synergistic photoprotective effect is achieved. Further by providing a source of epidermal retinyl esters to these embodiments, further protection against photosensitivity is also achieved.

In some embodiments of the serum, calcium ketogluconate is present. This is beneficial as the presence of calcium ketogluconate in the serum helps stimulate the production of hyaluronic acid and elastin in the user's skin, thereby increasing firmness, providing for more even tones, and preventing against the formation of wrinkles. Embodiments of the serum also exist which include tetrasodium glutamate diacetate, a chelating agent which binds to metallic ions thereby protecting the user's skin from the harmful effects of such ions. Embodiments exist where the serum also include phenylethyl which provides a natural and pleasing fragrance to the serum.

Embodiments of the serum can also include rosemary extract which is beneficial due to its high bioavailability profile.

Embodiments of the serum exist where the serum includes water, glycerin, sodium ascorbyl phosphate, calcium ketogluconate, rosemary extract, marigold flower extract, pinus pinaster bark extract, panax ginseng root extract, niacinamide, kojic acid, resveratrol, sodium hyaluronate, chondrus chrispis extract, bakuchiol, silybum marianum fruit extract, turmeric root extract, licorice root extract, Camellia sinensis leaf extract, bambusa vulgaris extract, beta-carotene, tocopherol, tetrasodium glutamate diacetate, squalane, caesalpinia spinosa gum, acacia senegal gum, xanthan Gum, phenethyl alcohol, and ethylhexyglycerin.

The present disclosure contemplated a number of embodiments of an oral nutritional supplement which promote the body's natural photoprotective properties when consumed on a regular basis. These embodiments can be consumed as a powder or included as some sort of tablet. Additionally, the embodiments of the nutritional supplements exhibit highly synergistic and unexpected benefits to users when combined with a regimen of regular application of embodiments of the serum. Moreover, when taken in connection with the regular application of embodiments of the cream and the embodiments of these serums, exceptionally unexpected synergistic photoprotective effects are imparted on the user.

Some embodiments of the e nutritional supplement can include methylsulfonylmethane, which helps regulate the genomic expression of key genes responsible for skin health as well as the slowdown in the signs of aging in humans. When embodiments of the nutritional supplement are taken regularly, the inclusion of methylsulfonylmethane will dramatically slow down such signs and promote healthy skin in the user. Specifically, consumption of these embodiments will provide significant improvements in reducing crow's feet and improving skin firmness, tone, and texture. Embodiments of the nutritional supplement can also include bamboo extract. In addition to the benefits disclosed herein when applied topically, the oral consumption of the embodiments of the nutritional supplement which contain bamboo extract also provide photoprotective benefits to the user. Specifically, the consumption of these embodiments reduces inflammation in the user and promotes cell rejuvenation and cell growth. Additionally, this oral consumption provides benefits in the amount of UV a user's skin can safely absorb, due to both the flavonoid content and polyphenol content present in these embodiments, and also provides the benefit of stimulating DNA repair while preventing further skin damage due to UV radiation exposure.

Embodiments of the nutritional supplement can also include fucoidan or wakame powder. These embodiments, when regularly consumed orally, reduce wrinkle formation, skin edema, and neutrophil recruitment into the photo-damaged lesions. By reducing skin inflammation, improving overall blood flow, and stimulating cell repair, these embodiments allow a user to reduce the appearance of any scars and/or wrinkles present on their skin. Additionally, these embodiments are high in vitamins and fatty acids, including various dietary minerals, omega-3, omega-6 fatty acids, and ascorbic acid. Embodiments of the nutritional supplement which include fucoidan will suppress matrix metalloproteinase-1 (MMP-1) activity, which is beneficial as MMP-1 is an enzyme which results in collagen damage and photoaging of skin due to UV irradiation. By including sulfated polysaccharides which are present in fucoidan, the expression of NF-κB is downregulated, which diminishes MMP-1 expression. Embodiments exist where low-molecular weight fucoidan are used, but other embodiments of high-molecular weight fucoidan also exist.

Various embodiments of the nutritional supplement disclosed herein include polypodium leucotomos extract, which promotes antioxidant activity in a user who regularly consumes these embodiments. As polypodium leucotomos extract contains caffeic acid and ferulic acid in addition to chlorogenic acid, coumaric acid, vanillic acid, and ferulic acid, regulation of numerous dermal responses to ultraviolet-induced erythema is achieved. In these embodiments, the user's natural ability to neutralize superoxide anions, lipid peroxides, and hydroxyl radicals are enhanced. Moreover, the users of these embodiments will exhibit lower levels of UV-induced COX-2 expression, p53 suppressor gene mutations, cyclobutene pyrimidine dimers, epidermal proliferation, sunburn cells, and inflammatory infiltrate will be enhanced. Additionally, as these embodiments promote antioxidant activity, the body of the user is more able to readily intercept inflammatory UV radiation as well as numerous cytotoxins. Moreover, these embodiments create a histological response on the overall reduction of angiogenesis, photo carcinogenesis, and solar elastosis, while also providing improvements to cell membrane integrity and elastin expression in the user.

Nutritional supplements can also include L-glutathione, which result in said nutritional supplement possessing antiaging, antimelanogenic, antioxidant, and anticancer properties when consumed regularly. Such consumption has an inhibitory effect on melanogenesis activity and oxidative stress caused by UV-B radiation. As noted herein, rosemary extract or rosemary powder has high bioavailability providing a benefit to embodiments of the nutritional supplement that include it, additionally enhancing the antioxidative properties of said nutritional supplement via such inclusion.

Embodiments of the nutritional supplement can also include marigold extract 2%. As marigold extract is high in antioxidants such as polyphenols, flavonoids, and carotenoids contents, the user's skin is protected from oxidative stress and cellular regeneration is promoted therein due to the consumption of these embodiments. Additionally, these embodiments necessarily have a high content of terpenoids and provide the protective effects thereof to the user, while also boosting collagen and elastin production in the user. In embodiments of the nutritional supplement, beta carotene 1% can also be included.

The nutritional supplement can also include vitamin B3 in different forms, including niacinamide. When present in embodiments of the nutritional supplement, niacinamide helps build proteins in the user's skin as well as help the skin retain moisture to prevent environmental damage. These embodiments also allow a user's body to naturally rebuild healthy skin cells, while also providing protection these cells from UV radiation. Additionally, regular consumption of these embodiments helps the skin of a user to develop ceramides, which create a healthy lipid barrier thereon, assisting the user skin in retaining moisture. Moreover, the regular consumption of these embodiments helps reduce inflammation in the user's skin caused by UV radiation, environmental pollution, and certain toxins. These embodiments, when consumed, also help regulate the amount of oil produced by the user's the sebaceous glands, thereby preventing an overproduction of sebum which can cause both acne and other breakouts while also minimizing the pore appearance of the user. Finally, these embodiments are also effective in lightening hyperpigmentation and is likely due to its ability to increase collagen production.

Various embodiments of the nutritional supplement can include fucoxanthin. Such embodiments benefit the user be reducing oxidative stress by scavenging reactive oxygen special. Photosynthetic pigments present in fucoxanthin mitigate the harmful effects associated with UV irradiation by dissipating the excess energy as heat and returns to the initial ground state. Such embodiments of the nutritional supplement will also stimulate filaggrin promoter activity in UV radiation-induced sunburn, as well as promote skin barrier formation through the induction of UV-sensitive gene expression.

Embodiments of the nutritional supplement also exist which contain purslane extract, which provides anti-aging properties to said nutritional supplements. Such embodiments containing purslane extract have potent protective effects against skin photoaging by regulating the miR-138-5p/Sirt1 axis and should be used as a natural product for skin care. These embodiments also contain KCl, K₂SO₄, KNO₃, nicotinic acid, saponin, vitamins A, B, and C, noradrenaline, 1-noradrenaline, dopamine, and dopa all of which have high antioxidant biological activity. These embodiments also contains flavonoids, which provide for further photoprotective activity by absorbing UV-radiation with wavelengths in the 240 nm-290 nm range, as well as up toe 550 nm, via the covalent bonds in their structure. Moreover, such embodiments reduce apoptotic cell death of human fibroblasts and keratinocytes after UVB irradiation, by depolarizing the mitochondrial membrane. These embodiments also provide to the user a high amount of micronutrients such as potassium, phosphorus acid, leucine, alanine, lysine, valine, glycine, and isoleucine.

Similarly to embodiments of the serum in accordance of the present disclosure, embodiments of the nutritional supplement disclosed herein can include red panax ginseng, which will increase the antioxidant and anti-inflammatory properties of the user's skin. This is achieved by not only inhibiting inflammation but also by reducing oxidative damage to the user's cells thereby limiting exposure to radiation which causes the skin to age. Embodiments of the nutritional supplement can also include polyphenols derived from green tea extract, vitamin E, and/or vitamin D3 all of which provide antioxidizing properties to the user's skin when regularly consumed orally. Embodiments of the nutritional supplement exist which contained Apigenin, such as apigenin 98% which provides anti-inflammatory, antiviral, antioxidant, and anticancer properties to the embodiments which contain it. In some embodiments of the nutritional supplement, pine bark extract is included to help promote collagen production, reduce irritation, and to provide antioxidizing properties to the embodiments. Embodiments of the nutritional supplement exist that include astaxanthin and/or lycopene 10%.

In embodiments of the nutritional supplement which contain high amounts of polyphenols, both prevention and reduction in the sigs of photoaging are achieved. Specifically, decreased trans-epidermal water loss (TEWL), increased skin elasticity, increased collagen formation, and decreased facial pigmentation are all effects which occur on the skin of the user.

Development of the embodiments described herein included combining various herbs, extracts, and vitamins, and encountering unpredictable factors during the formulation process. Embodiments of the nutritional supplement can be contained within an organic rice hull capsule, which is suited for accommodating larger particle sizes of the ingredients.

The development of the proprietary combination of ingredients used in embodiments of the nutritional supplement was not a straightforward process. It involved numerous attempts and refinements to identify the proper balance of specific active ingredients, dosing, and delivery format. The success of this formulation lies in its unique combination of ingredients, which, when combined at specific dosages, exhibit synergistic effects, creating additional protective mechanisms for skin health.

Moreover, the efficacy of the embodiments of the nutritional supplement are significantly enhanced when the embodiments of the serum and/or the embodiments of the cream are used in conjunction. The embodiment of the cream is specifically designed to augment the efficacy of the embodiments of the nutritional supplement, and vice versa. This synergistic effect is achieved through the careful selection of complementary active ingredients in both components. Additionally, the dosing of the various skin protective compositions are meticulously calibrated to ensure optimal efficacy, crucial for the effectiveness of each individual product and for their combined impact when used together. This approach ensures that the synergy between the disclosed skin protective compositions is maximized, offering an advanced level of skin protection and care to the user.

For purposes of the present disclosure, unless specifically disclaimed, the singular includes the plural and vice-versa, the words “and” and “or” shall be both conjunctive and disjunctive, the words “any” and “all” shall both mean “any and all”.

Moreover, any components or materials can be formed from a same, structurally continuous piece or separately fabricated and connected.

Unless otherwise stated, the foregoing alternative examples are not mutually exclusive, but may be implemented in various combinations to achieve unique advantages. As these and other variations and combinations of the features discussed herein can be utilized without departing from the subject matter defined by the claims, the foregoing description of the embodiments should be taken by way of illustration rather than by way of limitation of the subject matter defined by the claims. In addition, the provision of the examples described herein, as well as clauses phrased as “such as,” “including,” and the like, should not be interpreted as limiting the subject matter of the claims to the specific examples; rather, the examples are intended to illustrate only one of many possible embodiments. Further, the same reference numbers in different drawings can identify the same or similar elements.

1. A method of providing photoprotection to a human user, comprising the steps of:
orally consuming, by the human user, one or more tablets configured to promote the human user's systemic protection against the effects of radiation in the ultraviolet, visible, and infrared spectra;
applying topically, by the human user, a serum configured to synergistically to promote the human user's systemic protection against the effects of radiation in the ultraviolet, visible, and infrared spectra in combination with the one or more tablets; and
repeating, by the human user, the steps at least once per day for a predetermined amount of time.

2. The method of claim 1, the repeating step being preceded by the step of:
applying topically, by the human user, a cream configured to absorb an amount of radiation in the ultraviolet, visible, and infrared spectra.

3. The method of claim 1, wherein the one or more tablets each comprise:
up to 2000 mg of methylsulfonylmethane,
up to 600 mg of bamboo extract,
up to 600 mg of fucoidan,
up to 5000 mg of purslane extract,
up to 5000 mg of polypodium leucotomos,
up to 250 mg of L-glutathione,
up to 250 mg of rosemary powder, and
up to 250 mg of marigold extract.

4. The method of claim 3, the one or more tablets each further comprising:
up to 200 mg of beta carotene 0.1% to 5%,
up to 70 mg of niacinamide,
up to 50 mg of resveratrol 10% to 30%,
up to 50 mg of red panax ginseng,
up to 50 mg of green tea 90% to 99% polyphenols,
up to 40 mg of selenium citrate 0.1%-5%,
up to 30 mg vitamin E,
up to 30 mg of pine mark extract,
up to 30 mg of mulberry fruit powder,
up to 20 mg of vitamin D3,
up to 20 mg of apigenin 90% to 99%,
up to 10 mg of lycopene 1% to 20%,
up to 10 mg of quercetin, and
up to 5 mg of luteolin.

5. The method of claim 4, the serum comprising:
glycerin,
squalane,
resveratrol,
bakuchiol,
silybum marianum fruit extract,
niacinamide,
mixed tocopherols,
kojic acid,
sodium ascorbyl phosphate,
sodium hyaluronate,
chondruis crispus extract,
tumeric extract,
licorice extract,
green tea extract,
brazilian bamboo extract,
pine bark extract,
red panax ginseng extract,
marigold extract,
beta carotene,
calcium ketogluconate,
tetrasodium glutamate diacetate,
rosemary extract,
caesalpinia spinosa gum,
acacia senegal gum,
xanthan gum,
phenethylalcohol, and
ethylhexyglycerin.

6. The method of claim 5, the serum further comprising water.

7. The method of claim 6, the water being purified water.

8. The method of claim 2, the cream comprising:
zinc oxide up to 25%,
C₁₂-C₁₅ alkyl benzoate,
one or more jojoba esters,
one or more triglycerides,
polyglyceryl-4 diisostearate/polyhydroxystearate/sebacate,
propanediol,
niacinamide,
methylpropanediol,
coco-caprylate,
polyglyceryl-2,
Helianthus annuus extract R,
rosemary extract,
tocopherol,
allantoin,
Oryza sativa bran extract,
caprylyl glycol,
polyglyceryl-3,
hydrogenated lecithin, and
tridecyl salicylate.

9. The method of claim 8, the cream further comprising silica, nylon-12, bisabolol, tetrasodium glutamate diacetate, phenylpropanol, sodium chloride, and sodium hydroxide.

10. A system for promoting a human's systemic protection against the effects of radiation in the ultraviolet, visible, and infrared spectra, comprising:
one or more tablets, the one or more tablets each comprising:
up to 2000 mg of methylsulfonylmethane,
up to 600 mg of bamboo extract,
up to 600 mg of fucoidan,
up to 500 mg of purslane extract,
up to 500 mg of polypodium leucotomos,
up to 250 mg of L-glutathione,
up to 250 mg of rosemary powder, and
up to 250 mg of marigold extract; and
a serum, comprising:
glycerin,
squalane,
resveratrol,
bakuchiol,
silybum marianum fruit extract,
niacinamide,
mixed tocopherols,
kojic acid,
sodium ascorbyl phosphate,
sodium hyaluronate,
chondruis crispus extract,
tumeric extract,
licorice extract,
green tea extract,
brazilian bamboo extract,
pine bark extract,
red panax ginseng extract,
marigold extract,
beta carotene,
calcium ketogluconate,
tetrasodium glutamate diacetate,
rosemary extract,
caesalpinia spinosa gum,
acacia senegal gum,
xanthan gum,
phenethylalcohol, and
ethylhexyglycerin.

11. The system of claim 10, the one or more tablets each further comprising,
up to 200 mg of Beta Carotene 0.1% to 5%,
up to 70 mg of niacinamide,
up to 50 mg of resveratrol 10% to 30%,
up to 50 mg of red panax ginseng,
up to 50 mg of green tea 90% to 99% polyphenols,
up to 40 mg of selenium citrate 0.1%-5%,
up to 30 mg vitamin E,
up to 30 mg of pine mark extract,
up to 30 mg of mulberry fruit powder,
up to 20 mg of vitamin D3,
up to 20 mg of apigenin 90% to 99%,
up to 10 mg of lycopene 1% to 20%,
up to 10 mg of quercetin, and
up to 5 mg of luteolin.

12. The system of claim 10, further comprising:
a cream, comprising:
zinc oxide up to 25%,
C₁₂-C₁₅ alkyl benzoate,
one or more jojoba esters,
one or more triglycerides,
polyglyceryl-4 diisostearate/polyhydroxystearate/sebacate,
propanediol,
niacinamide,
methylpropanediol,
coco-caprylate,
polyglyceryl-2,
elianthus annuus extract R,
rosemary extract,
tocopherol,
allantoin,
Oryza sativa bran extract,
caprylyl glycol,
polyglyceryl-3,
hydrogenated lecithin, and
tridecyl salicylate.

13. The system of claim 12, the cream further comprising silica, nylon-12, bisabolol, tetrasodium glutamate diacetate, phenylpropanol, sodium chloride, and sodium hydroxide.

14. The system of claim 13, the serum further comprising water.

15. The system of claim 14, the water being purified water.

16. A method of providing photoprotection to a human user, comprising the steps of:
applying topically, by the human user, a serum configured to synergistically to promote the human user's systemic protection against the effects of radiation in the ultraviolet, visible, and infrared spectra in combination with the one or more tablets;
applying topically, by the human user, a cream configured to absorb an amount of radiation in the ultraviolet, visible, and infrared spectra;
orally consuming, by the human user, one or more tablets configured to promote the human user's systemic protection against the effects of radiation in the ultraviolet, visible, and infrared spectra; and
repeating, by the human user, the steps at least once per day for a predetermined amount of time.
